(19) United States
(12) Patent Application Publication
(10) Pub. No.: US 2019/0104980 A1
(43) Pub. Date: Apr. 11, 2019

(54) NON-CONTACT APPARATUS AND METHOD FOR CAPTURING SKIN SURFACE IMAGE DATA

(71) Applicant: UNIVERSITY OF THE WEST OF ENGLAND, BRISTOL, Bristol (GB)

(72) Inventors: Abdul FAROOQ, Bristol (GB); Melvyn SMITH, Bristol (GB); Lyndon SMITH, Bristol (GB)

(73) Assignee: UNIVERSITY OF THE WEST OF ENGLAND, BRISTOL, Bristol (US)

(21) Appl. No.: 16/091,550

(22) PCT Filed: Apr. 5, 2017
(86) PCT No.: PCT/EP2017/058128

§ 371 Date: Oct. 5, 2018

(30) Foreign Application Priority Data
Apr. 6, 2016 (GB) 1605894.3

(51) Int. Cl. A61B 5/00; G06T 7/586

(52) U.S. Cl. CPC A61B 5/444; A61B 5/0064; G06T 7/586; A61B 5/0077; A61B 5/0037

(57) ABSTRACT

A non-contact skin imaging device for capturing 2D and 3D textural data from a skin surface using a photometric stereo technique in which a skin surface position detector is arranged to sense when the skin surface is in the optimal position for the 2D and 3D textural data to be collected. The device may comprise an optical range finder for determining a position of the skin surface, whereby capture of photometric stereo image data can be automatically triggered when the skin surface is in the optimal position. With this arrangement, a decision to capture the photometric stereo image data can be taken without the input of a human user.

### CROSS-REFERENCE TO RELATED APPLICATIONS

This is a U.S. national phase application under 35 U.S.C. § 371 of International Patent Application No. PCT/EP2017/058128, filed Apr. 5, 2017, and claims benefit of priority to British Patent Application No. 1605894.3, filed Apr. 6, 2016. The entire contents of these applications are hereby incorporated by reference.

### FIELD OF THE INVENTION

The invention relates to a device for capturing image data from a skin surface using photometric stereo (PS) techniques. In particular, the invention relates to a device (and a method of operating such a device) that can capture such image data automatically upon detecting that the skin surface is in an optimal position without requiring contact between the device and skin surface.

### FIELD OF TECHNOLOGY

Human skin exhibits complex textures in both 3D and 2D. A facility for recovering such texture data with good accuracy and repeatability would provide useful information in various fields. For example, in the healthcare field, any changes in pigmented lesions are of interest, since they can provide an indication that the lesion is becoming cancerous.

### BACKGROUND

Currently medical practitioners do not have access to devices that enable them to accurately and repeatably measure such changes. In fact, they often have nothing other than a rule for measuring skin lesions.

There are accepted heuristics that are intended for determining if a given lesion is suspicious—such as the ‘ABCD rules’. Unfortunately however, since there is no way to reliably capture lesion characteristics, objective and quantitative ABCD analyses cannot currently be achieved. Also, the current method of capturing the appearance of a lesion in hospitals is to have it photographed using a conventional digital camera. Since the position of the camera and the lights relative to the skin are likely to change considerably between two photographs of the same lesion taken at different times, the lesion can appear different even when it has not changed; and this prevents effective detection of change.

In healthcare, skin cancer is becoming an increasingly common condition, however GPs receive little training in recognising it since it used to be a rare disease in the UK, and so tend to over-refer patients to skin specialists. As a result, hospital pigmented lesion clinics are generally overcrowded with patients, the majority of whom do not have suspicious lesions. This results in the risk of a patient with a suspicious lesion being missed in the busy conditions, which is very serious because a skin cancer such as melanoma is a potentially fatal disease which has to be detected and treated as early as possible for the best chance of a good long-term prognosis.

There are devices that employ frequency based techniques for analysing lesions. For example, spectrophotometric intracutaneous analysis (SIAscopy) can be used to detect substances present at the surface of lesions, for inferring the possible presence of cancer. However, this approach depends upon models which have been questioned by researchers who have reported poor performance in differentiating between different types of lesion.

Another device that can be used for studying lesions is the dermatoscope. Here a window is pressed against an illuminated lesion to allow a doctor to view structure below the surface. The drawback to this approach is that it requires a relatively high level of training in its use, which most doctors do not have, thereby preventing its more widespread use.

Therefore, reliable recovery and analysis of 2D and 3D textures from skin lesions offers potential for assisting with early detection of suspicious lesions.

Another field where detection and analysis of 3D skin features is of interest is cosmetics. Many products are marketed as being able to assist with apparently slowing the aging process by reducing the size of wrinkles. If a device were available that could accurately measure wrinkle size it could be used to objectively evaluate the effectiveness of such products. Also, a device that could easily recover the true colour of skin could be used by individuals for planning and customizing their use of cosmetics. For example, a person with Rosacea may wish to employ a foundation makeup that provides the best chance of effectively masking the condition. Detection of true colour would assist with determination of the optimal colour of foundation to be applied.

It has been proposed by the present inventors, among others, to make use of machine vision techniques to obtain 2D and 3D skin texture information for the detection of melanoma [1, 2].

WO 2010/097218 discloses an optical device for imaging and measuring characteristics of the topography of human skin using photometric stereo techniques. In this device, a plurality of illumination sources are arranged to illuminate the skin surface from different angles. Polarisers are used to eliminate specular reflection.

Photometric stereo (PS) is a machine vision technique for recovering 3D surface normal data (known as a ‘bump map’) and 2D reflectance data (known as albedo) from surfaces. Photometric stereo employs a number of lights in known locations and a single camera [3-6]. An image is captured when one of each of the lights is turned on in turn. The obtained images are processed and combined using a lighting model (such as Lambert's Law, which assumes that the brightness of a pixel at a point on the surface is proportional to the cosine of the angle between the vector from the point to the source and the surface normal vector at that point), in order to generate the bump map (i.e. a dense array of surface normals sometimes referred to as 2.5D data) and the albedo (an image of surface reflectance).

FIG. 1 shows a schematic view of an apparatus for performing photometric stereo measurements. A plurality of light sources (which are also referred to as illuminates) S1, S2, S3 are positioned above a surface 10 to be inspected, which lies in the field of view of a camera 12. The position of the light sources relative to the surface are known accurately, so that an incident light vector from each source is known for each point on the surface. To fully recover the orientation of a surface normal N in a three-dimensional coordinate system (e.g. formed by axes X, Y, Z), a minimum of three light sources are required to be arranged in a manner whereby, between them, the incident vectors provide components along all three axes.

Photometric stereo differs from the conventional imaging techniques mentioned above in that the captured images are combined using the lighting model to generate the bump map and albedo (on which further assessment is based), whereas the conventional techniques simply compare raw image data.

### SUMMARY

At its most general, the present invention proposes a device for capturing 2D and 3D textural data from a skin surface using a photometric stereo technique in which a skin surface position detector is arranged to sense when the skin surface is in the optimal position for the 2D and 3D textural data to be collected.

According to one aspect of the invention there is provided a non-contact skin imaging device comprising: a photometric stereo imaging apparatus arranged to capture photometric stereo image data from a skin surface; an optical range finder arranged to determine a position of the skin surface; and a controller in communication with the optical range finder, the controller being arranged: to judge whether or not the skin surface is in an optimal position for capturing the photometric stereo image data, and upon judging that the skin surface is in the optimal position, to automatically trigger capture of the photometric stereo image data. With this arrangement, the decision to capture the photometric stereo image data can be taken without the input of a human user. The controller therefore comprises a hardware-based entity, e.g. comprising a processor capable of executing software instructions to carry out the relevant steps.

The photometric stereo imaging apparatus may be conventional. The photometric stereo imaging apparatus may comprise an image capture device (e.g. a digital camera) and an illumination array comprising a plurality of illuminates (e.g. selectively activatable radiation sources capable of emitting visible and/or infra-red radiation) to illuminate a field of view of the image capture device from different directions. The location of each illuminate relative to the image capture device is known so that the incident light vector at each point on the surface is known.

The illumination array may comprise a ring of light sources mounted around the periphery of the field of view of the image capture device. The light sources can be any suitable point-like source, e.g. LEDs or the like.

The optical range finder may be arranged to work in conjunction with the image capture device using the principles of triangulation. For example, the optical range finder may comprise a collimated light source mounted in a fixed position relative to the image capture device, the collimated light source being arranged to emit a collimated light beam through the field of view of the image capture device. The direction of the collimated light beam through the field of view is known, so the position at which is intersects a surface in the field of view is related to the distance of that surface from the image capture device.

The optical range finder may comprise a plurality of (e.g. three) collimated light sources mounted in different respective fixed positions relative to the image capture device, wherein the plurality of collimated light source are arranged to emit a plurality of collimated light beams through the field of view of the image capture device. Having more that one point of intersection with the surface permits information about the orientation of the surface (i.e. its angle relative to the image capture device) to be determined. This information may also be used by the controller to judge whether or not the skin surface is in an optimal position for capturing the photometric stereo image data.

The plurality of collimated light sources may be oriented so that the plurality of collimated light beams converge as they pass through the field of view of the image capture device. This can assist a user in moving the device relative to the skin surface so that it is in the optimal position. The plurality of collimated light beams may be arranged to intersect at a distance from the image capture device that corresponds to the optimal position.

The controller may be in communication with the image capture device to monitor a position at which the collimated light beam(s) intersect the skin surface, whereby the controller is arranged to judge whether or not the skin surface is in an optimal position for capturing the photometric stereo image data based on the position at which the collimated light beam(s) intersect the skin surface. For example, the controller may judge that the skin surface is in an optimal position for capturing the photometric stereo image data if the positions at which the collimated light beams intersect the skin surface are within a predetermined region.

The collimated light beams may project as spots or points on the skin surface. The controller may be arranged to judge that the skin surface is in an optimal position for capturing the photometric stereo image data if these points are spaced from each other by less than a threshold distance.

The collimated light source(s) may be arranged to emit a planar light beam, which projects as a line on the skin surface. These lines can be used to as an independent source of 3D surface profile data. The controller may be arranged to judge that the skin surface is in an optimal position for capturing the photometric stereo image data based on the position at which these lines intersect each other.

The controller may also be arranged to check that the device is held steady relative to the skin surface before the photometric stereo image data is captured. For example, the controller may be arranged to determine a rate of change of the position at which each collimated light beam intersects the skin surface, whereby the controller is arranged to judge that the skin surface is in an optimal position for capturing the photometric stereo image data if the rate of change of the positions at which the collimated light beams intersect the skin surface is less than a predetermined threshold.

The controller may comprise a field programmable gate array in communication with the image capture device. With this arrangement transformation and processing of the image data can be reduced or minimised, which speeds up the judgement process.

The device may be portable, e.g. powered by a battery and contained in a hand-held housing.

In another aspect, the invention provides a non-contact method of capturing photometric stereo image data of a skin surface, the method comprising: determining, using an optical range finder, a position of the skin surface within a field of view of an image capture device; judging whether or not the skin surface is in an optimal position for capturing the photometric stereo image data; and upon judging that the skin surface is in the optimal position, automatically triggering capture of the photometric stereo image data.

The method may include the functions carried out by the controller discussed above.

For example, the optical range finder may comprise a plurality of collimated light sources mounted in different respective fixed positions relative to the image capture device. In this example, the method may comprise emitting a plurality of collimated light beams through the field of view of the image capture device, and monitoring, by an image processing controller in communication with the image capture device, a position at which the collimated light beams intersect the skin surface. In this arrangement, judging whether or not the skin surface is in an optimal position for capturing the photometric stereo image data may be based on the position at which the collimated light beams intersect the skin surface. For example, judging whether or not the skin surface is in an optimal position for capturing the photometric stereo image data may comprise determining whether or not the positions at which the collimated light beams intersect the skin surface are within a predetermined region.

### BRIEF DESCRIPTION OF THE DRAWINGS

Examples of the invention are discussed below with reference to the accompanying drawings, in which:

FIG. 1 is a schematic view of an apparatus for performing photometric stereo measurements, discussed above;

FIG. 2 shows schematic front and side views of a skin image capture device that is an embodiment of the invention;

FIG. 3 is a schematic diagram showing a first configuration of illumination sources and camera capable of automatically capturing skin image data in an embodiment of the invention; and

FIG. 4 is a schematic diagram showing a second configuration of illumination sources and camera capable of automatically capturing skin image data in an embodiment of the invention.

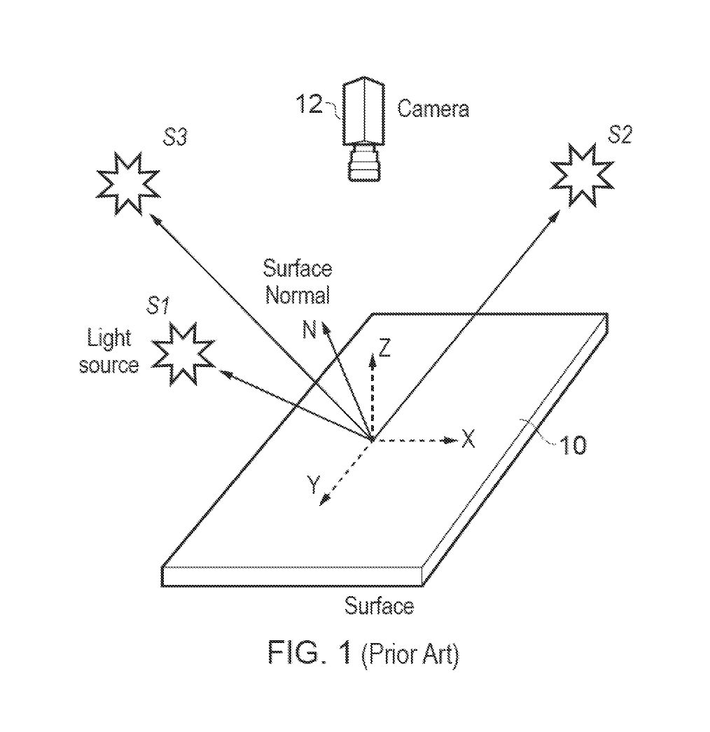

### DETAILED DESCRIPTION

The disclosure herein described a non-contact vision based method and device for automatically triggering capture of photometric stereo image data of a surface. The automatic triggering is based on sensing the range and/or the orientation of the surface with respect to the imaging capture device (e.g. camera). The method and device may find particular use on movable surfaces where it is desirable for there to be no contact with the entity being imaged. As explained above, the method and device of the invention is particularly advantageous for capturing images of skin.

Sensing the range of the surface (e.g. skin surface) may mean determining a separation between the surface and a camera in the device, and in particular between the surface and any focussing optics in the camera.

Sensing the orientation of the surface may mean determining an angle of the skin surface with respect to an optical axis of the camera.

The photometric image data may comprise a set of images of the surface captured under different light conditions. The invention may operate to automatically trigger capture of the image data when the skin surface is in an optimal position. The optimal position may be when the range and/or orientation of the surface is determined to lie within a certain predetermined band of values.

The invention enables recovery of high-resolution 3D and 2D data from the skin surface with high accuracy and good repeatability. The automatic triggering makes the device easy of use, whilst the non-contact nature of the method ensures that the technique is hygienic.

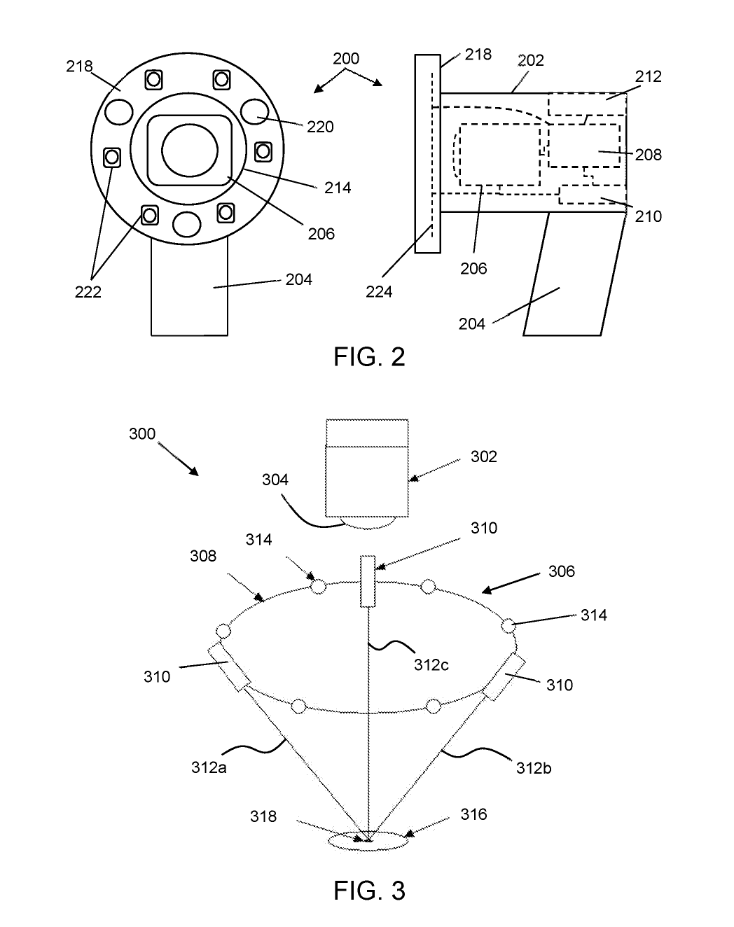

FIG. 2 shows schematic front and side views of a device 200 that is an embodiment of the invention. The device 200 comprises a housing 202 and a handle 204. The housing 202 and handle 204 may be made of suitably robust material for use in a clinical setting. They may be sterilisable. The housing 202 comprising a hollow main body that contains a image capture device 206 (e.g. digital camera), a controller 208, a power source 210 (e.g. battery), and a communications module 212. The front of the main body has an aperture 214 that is open so that a region in front of the device is in the field of view of the camera.

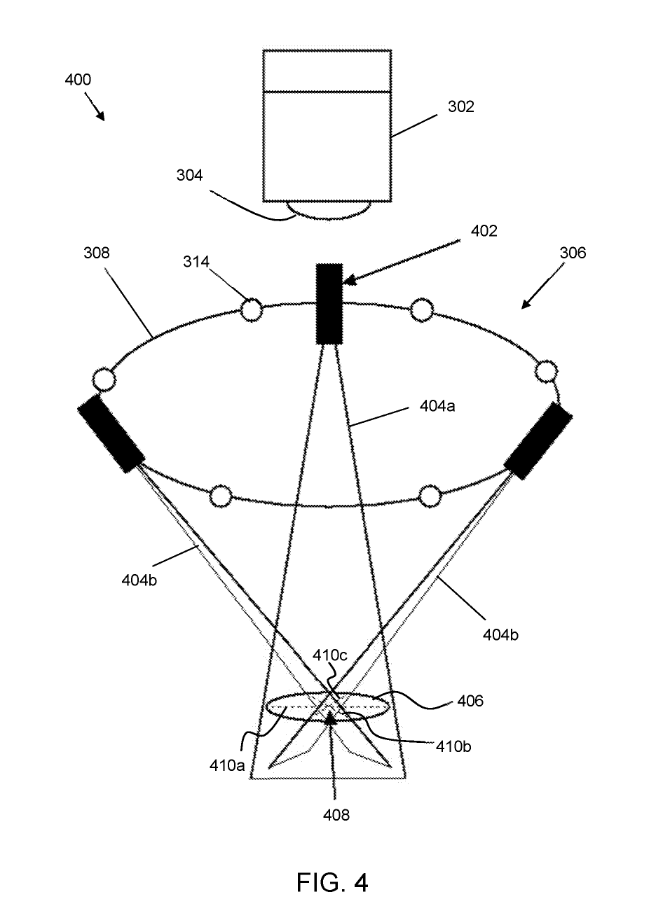

An illumination array 218 is arranged around the aperture at the front of the housing 202. In this example, the illumination array 218 is an annular body that has a plurality of illumination sources mounted therein. The plurality of illumination sources comprise one or more range finding light sources 220 and a plurality of photometric stereo light sources 222. The number and function of these components is discussed in more detail with reference to FIGS. 3 and 4. The illumination sources may be mounted on a circuit board 224 that is arranged to receive power from the power source 210 and control signals from the controller 208.

The image capture device 206 performs two operations. Firstly, during positioning of the device relative to a surface to be measured, the surface to be measured is illuminated using the range finding light sources 220, and the camera 206 captures images which are assessed to determine whether or not the surface is in an optimal position. Secondly, once the surface is in an optimal position, the camera 206 is used to capture photometric stereo image data. The controller 208 is arranged to control both of these operations. The steps involved are discussed in more detail with reference to FIGS. 3 and 4 below.

FIG. 3 presents one configuration 300 that is suitable for implementing the present invention.

The configuration 300 comprises a digital camera 302 with lens 304. In front of the camera there is an illumination array 306. In this example, the illumination array 306 comprises a plurality of illuminates disposed around a ring 308, which is located around the periphery of the camera's field of view. The plurality of illuminates themselves are preferably not visible in the camera's field of view. In other words, the ring 308 is positioned with respect to the camera so that the illuminates project light into the camera's field or view but are not themselves visible in the field of view.

In this example, the plurality of illuminates comprise three collimated light sources 310, e.g. comprising low-power lasers or LEDs, which are arranged to output respective collimated rays of light 312a, 312b, 312c. In this example, the collimated light sources 310 are equally spaced around the ring, but the invention need not be limited to this arrangement.

In addition to the collimated light sources 310, the plurality of illuminates also includes a set of light sources 314 for creating lighting conditions suitable for making photometric stereo measurements. In this example, the set of light sources 314 comprises six illuminates that are spaced around the ring 308. The six illuminates are equally spaced in this example, but the invention need not be limited to such a configuration.

The collimated light sources 310 are oriented relative to the camera to be suitable as range-finding reference beams. If a surface is positioned in the field of view of the camera, a set of light spots will be visible at the points where the collimated rays of light 312a, 312b, 312c meet that surface. If the position of each collimated light sources 310 relative to the camera and the direction of its respective collimated rays of light 312a, 312b, 312c is known, the distance of the surface from the camera can be determined based on the configuration of the set of light spots.

In one example, the collimated rays of light 312a, 312b, 312c extend in respective directions that converge towards an axis extending from the camera. The camera axis may be an optical axis of the lens 304 in the camera. In this example, the separation of the set of light spots is an indicator of the distance between the surface and the camera.

The collimated rays of light 312a, 312b, 312c may be arranged to intersect each other. In one example, the collimated light sources 310 are arranged so that the point of intersection is at a predetermined distance from the camera. The predetermined distance is preferably set to be the optimal location for a surface in order for the camera to capture photometric stereo images using the illuminates 314. The point of intersection may lie on the camera axis, but that is not essential.

In the above arrangement, a surface 316 (such as a skin lesion or the like) will be in an optimal position for capturing photometric stereo data when the collimated rays of light 312a, 312b, 312c form a single spot 318 on that surface 316. In this example, the collimated light sources 310 act as a guide to assist a user in positioning the camera 302 and illumination array 306 in the correct location relative to a surface 316. The separation of the light spots is a guide to distance along the camera axis (e.g. along a Z axis); the closer together the light spots the nearer to the optimal position. And the position of the set of light spots on the surface assists in locating the relevant part of the surface in the field of view of the camera (e.g. in an X-Y plane).

In order to automatically trigger capture of the photometric stereo image data, the camera 302 may be arranged to capture images of the set of light spots during positioning, e.g. in a continuous or quasi-continuous manner. The captured images may be analysed to identify light spots corresponding to the collimated rays of light 312a, 312b, 312c in the field of view. One or more properties of the identified light spots may then be used to determine whether or not the surface is within an acceptable range for capturing the photometric stereo image data. For example, the absolute separation between the identified light spots and the rate of change of that separation may be calculated. If it is determined that the separation falls below a predetermined threshold (corresponding to an optimal distance between the camera and surface) and that the rate of change of the separation is below a predetermined threshold (e.g. indicating that the camera is being held steady relative to the surface), the device may proceed to capture the photometric stereo image data.

In the example shown in FIG. 3 capture of the photometric stereo image data may be triggered when the collimated rays of light 312a, 312b, 312c intersect on the surface 316. In other example, some tolerance may be permitted, so that some degree of separation is permitted.

Where the point of intersection of the collimated rays of light 312a, 312b, 312c within the field of view of the camera is known, the analysis of the light spots can also be used to judge the orientation of the surface because the position of the light spots within the field of view can be used to triangulate the distance to the surface. Where three light spots are provided, it is possible to determine a plane on which those light spots lie, and hence an orientation of that plane relative to the camera axis. The angle of that plane relative to the camera axis and the rate of change of that angle may also be used to determine whether or not the surface is within an acceptable range for capturing the photometric stereo image data. For example, if it is determined that an angle between a direction normal to the plane and the camera axis falls below a predetermined threshold (corresponding to an optimal orientation between the camera and surface) and that the rate of change of that angle is below a predetermined threshold (e.g. indicating that the camera is being held steady relative to the surface), the device may proceed to capture the photometric stereo image data. In an alternative example, the angle information may be used to rectify the captured images, i.e. compensate for any orientation by manipulating the captured image data using known image processing techniques.

It is desirable for the automatic triggering determination to be processed as rapidly as possible. In one example, the analysis is performed by hardware associated with the camera itself. For example, a field-programmable gate array (FPGA) and on-board memory in the camera can be used to effectively perform the necessary analysis on temporarily held images, without requiring those images to be transferred for processing elsewhere. This arrangement may dramatically increase the speed at which the surface position is assessed and at which the photometric stereo image data capture can be triggered. Speeding up the assessment and triggering process minimises or eliminates the effect of movement of the surface, thereby improving the registration of the photometric stereo images and the quality of the subsequent 3D and 2D data captured.

The collimated rays of light 312a, 312b, 312c may have any beam cross-section shape. The set of light spots may be simple light points. However, in other example, they may be other projected patterns, e.g. circles, lines or other shapes. Using other patterns may assist in identifying the set of light spots in the field of view of the camera, and may also assist determining the orientation of the surface relative to the axis of the camera.

To capture the photometric stereo image data, a set of images of the surface is captured by the camera, with each image in the set having a different illumination condition. For example, there may be six images in the set, each image showing the surface when illuminated by a respective one of the light sources 314. However, the invention is not limited to this specific scenario. The set of images may contain more or fewer than six images. The surface may be simultaneously illuminated by two or more of the light sources 314.

The collimated light sources 310 may be switched off when the photometric stereo image data is captured, but this is not essential. In fact, it may be desirable for the collimated light sources 310 to remain activated in order to check that the surface does not move significantly while the photometric stereo image data is obtained.

The camera 302 may be any type of digital camera. To prevent movement of the surface from affecting the photometric stereo image data, the camera 32 is preferably capable of capturing multiple images at high speed, e.g. a burst mode or similar. The camera 302 and light sources 314 may be activated by a common controller that is arranged to coordinate the photometric stereo image data capture operation.

The camera 302 may operate in visible light and/or other wavelengths. For example, multispectral illumination could be employed, where each light source 304 is an LED that operates at a specific wavelength and narrow bandwidth. Infra-red (IR) wavelengths could be employed, with cameras exhibiting high sensitivity and extended performance into the IR (1200 nm).

Filters can be employed in the camera to enable multiple photometric stereo images to be captured simultaneously. The filters match the wavelengths of the light sources, so it becomes possible to recover surface data.

Further information about the technique of performing analysis of a skin surface using photometric stereo image data is presented below with reference to FIG. 4.

After the photometric stereo image data is captured, it can be transferred (e.g. wirelessly via Bluetooth® or the like) to the host computer for further processing, heuristic analysis, visualisation and wider dissemination.

FIG. 4 presents another configuration 400 that is suitable for implementing the present invention. Features in common with FIG. 3 are given the same reference numbers and are not described again.

In this example, the illumination array 306 comprises three planar light beams sources 402, e.g. comprising low-power lasers or LEDs in conjunction with line generating optics (e.g. a cylindrical lens or the like), which are arranged to output respective planar light beams 404a, 404b, 404c. In this example, the planar light beams sources 402 are equally spaced around the ring, but the invention need not be limited to this arrangement.

This configuration again employs three collimated light sources (e.g. lasers or LEDs) for the purpose of detecting the range and orientation of the surface to be measured, e.g. a skin surface having a lesion thereon. In this example, each of the collimated light source is arranged to output a planar light beam, which forms a line when it intersect with the surface to be measured. The planar light beam can be formed using any known technique. For example, one possible implementation would employ a cylindrical lens (with a profile arranged to give a ‘flat top’ intensity distributions along the laser line). The ‘fan angle’ of each beam, i.e. the angle of lateral spread in the plane of the beam may be, for example, between 10 to 20 degrees.

Similarly to the configuration shown in FIG. 3, the light sources 402 are arranged so that the planes of the planar light beams are oriented relative to the camera axis to be suitable as range-finding reference beams. If a surface is positioned in the field of view of the camera, a set of lines will be visible at the points where the planar light beams 404a, 404b, 404c meet that surface. If the position of each light source 402 relative to the camera and the direction of its respective planar light beam 404a, 404b, 404c is known, the distance of the surface from the camera can be determined based on the configuration of the set of light spots.

In the example shown, the lights sources 402 are arranged so that the planar light beams intersect in the field of view of the camera. The three planar light beams 404a, 404b, 404c are therefore projected onto the surface at known angles.

The three planes of light create three lines of light 410a, 410b, 410c at the point where they intersect the surface 406 (see dotted lines in FIG. 4).

The point 408 at which the lines 410a, 410b, 410c intersect may be set to be at the optimum distance from the camera for capturing photometric stereo image data. Thus, then the lines are visible on the surface 406, they act as a guide to facilitate positioning the camera relative to the surface in an optimum location.

As discussed above, the camera may be arrange to monitor the appearance of the lines on the surface. In most positions, the lines 410b, 410c will cross the line 410a at difference points. The points will get closer together until they meet when the surface is in the position shown in FIG. 4. The device may monitor the separation of these points and the rate of change of that separation. If the separation is judged to be less than a predetermined threshold and the rate of change of the separation is below a threshold (which indicates that the camera is held steady relative to the surface), the device can be arranged to automatically trigger capture of the photometric stereo image data as discussed above.

In one example, the photometric stereo image data may be triggered when the three lines intersect at a single point as shown in FIG. 4.

The lines 410a, 410b, 410c may also be used to obtain 3D profile data about the surface being measured. Since the angles of the laser planes of light are known, triangulation can be employed to accurately find the distance, i.e. height of the skin surface, at each point along the lines 410a, 410b, 410c shown in FIG. 4. This is important because it provides functionality that is complementary to the photometric stereo image data. Photometric stereo provides excellent capabilities for recovering the 3D surface (gradients) of the surface in high-resolution (i.e. as a dense array of surface normals). However, a 3D surface relief is difficult to recover accurately because the process of integrating the gradients can cause errors to build up. However, if accurate 3D height data from the three laser lines is obtained, it can be used as ground truth height data to remove these errors. In this way the technique of the invention can provide a convenient and low-cost method of accurately recovering the overall morphology of a lesion as well as its 3D texture and true colour. The capability for accurate 3D shape recovery would be expected to prove useful for 3D segmentation of lesions, or when endeavouring to develop 3D heuristics for assisting with identification of suspicious lesions. Such 3D heuristics would be expected to be analogous to the 2D ‘ABCD’ rules and complementary to them. Rather than replacing the ABCD rules they could be used in addition to them, to provide additional indicators of possible skin cancer. This technique would also be useful for measuring (size and volume) of skin wounds in 3D. The triangulated height data from the laser lines would also assist with image registration. If a slight relative movement between the skin and the device were to occur between successive captured images in the photometric stereo image data, then this could be detected and quantified through analysis of the change in the laser line height profiles. This displacement information could then be used to eliminate the movement, thereby assisting with good image registration that would help with generating the best possible 2D and 3D lesion data.

The present invention is an automatic trigger mechanism for a method and device arranged to utilise photometric stereo techniques to measure the 3D (texture and morphology) and 2D (pigment) characteristics of the skin surface, including lesions (moles).

In addition to the automatic triggering functionality discussed above, the device may comprise one or more of the following features.

The device may incorporate multi-spectral illumination, thereby enabling application of multi-spectral techniques such as SIAscopy.

The device may incorporate polarising filters and/or infra-red illumination to enable use of techniques such as dermoscopy where structure beneath the surface can be detected. By employing multiple wavelengths of infra-red illumination, structure at different distances below the surface can be examined.

Normally three illuminates are used when capturing photometric stereo image data. However, it has been found beneficial to use more than three, e.g. 6 or more, illumination to enable data recovery from any convex object and also provides redundancy that can assist with elimination of artefacts such as shadows and highlights.

Any suitable data analysis technique can be used to assess the captured photometric stereo image data. For example, neural networks or other machine learning technique can be used to providing quantitative and qualitative information on 3D and 2D skin characteristics.

The photometric stereo image data captured by the device of the invention can comprise 3D surface normal data (the ‘bump map’) and 2D surface reflectance or pigment data (the ‘albedo’). Photometric stereo employs a number of lights located in known directions and one camera. An image is captured with each of one of the lights turned on, one at a time. The resulting images are processed and combined with a lighting model such as Lambert's Law (which models the brightness of a pixel as being proportional to the cosine of the angle between the surface normal at that point and the lighting vector), in order to generate the bump map (a dense array of surface normal over the image) and the albedo (an image of the surface reflectance which gives the surface pigment in true colour).

In summary, the proposed non-contact arrangement for triggering photometric stereo image capture is intended to improve the ease and speed with which a device can be used (even by a layperson), and to provide improved hygiene and reduced chance of disease transfer. Obviating the need for contact with the skin should improve the chances of being able to use the device to access wounds in locations on the body that might not be accessible for contact based devices. Finally, the employment of planes of laser light with triangulation, as shown in FIG. 4, is expected to increase the accuracy of the 3D skin shape recovery, thereby further increasing the utility of the device.

One particularly advantageous use of the invention may be to image lesions on the tongue. At present it is difficult to obtain useful images in this context. The present invention may provide a non-contact solution that can minimise the risk of contamination whilst ensuring repeatability so that changes in the lesion over time (which are a critical indication of cancer) can be measured.

1. A non-contact skin imaging device comprising:
a photometric stereo imaging apparatus arranged to capture photometric stereo image data from a skin surface;
an optical range finder arranged to determine a position of the skin surface; and
a controller in communication with the optical range finder, the controller being arranged:
to judge whether or not the skin surface is in an optimal position for capturing the photometric stereo image data, and
upon judging that the skin surface is in the optimal position, to automatically trigger capture of the photometric stereo image data.

2. A non-contact skin imaging device according to claim 1, wherein the photometric stereo imaging apparatus comprises:
an image capture device; and
an illumination array comprising a plurality of illuminates arranged to illuminate a field of view of the image capture device from different angles.

3. A non-contact skin imaging device according to claim 2, wherein the illumination array comprises a ring of light sources mounted around the periphery of the field of view of the image capture device.

4. A non-contact skin imaging device according to claim 2, wherein the optical range finder comprises a collimated light source mounted in a fixed position relative to the image capture device, the collimated light source being arranged to emit a collimated light beam through the field of view of the image capture device.

5. A non-contact skin imaging device according to claim 4, wherein the optical range finder comprises a plurality of collimated light sources mounted in different respective fixed positions relative to the image capture device, wherein the plurality of collimated light source are arranged to emit a plurality of collimated light beams through the field of view of the image capture device.

6. A non-contact skin imaging device according to claim 5, wherein the plurality of collimated light sources are oriented so that the plurality of collimated light beams converge as they pass through the field of view of the image capture device.

7. A non-contact skin imaging device according to claim 6, wherein the plurality of collimated light beams are arranged to intersect at a distance from the image capture device that corresponds to the optimal position.

8. A non-contact skin imaging device according to claim 4, wherein the controller is in communication with the image capture device to monitor a position at which the collimated light beam(s) intersect the skin surface, whereby the controller is arranged to judge whether or not the skin surface is in an optimal position for capturing the photometric stereo image data based on the position at which the collimated light beam(s) intersect the skin surface.

9. A non-contact skin imaging device according to claim 7, wherein the controller is in communication with the image capture device to monitor a position at which each collimated light beam intersects the skin surface, whereby the controller is arranged to judge that the skin surface is in an optimal position for capturing the photometric stereo image data if the positions at which the collimated light beams intersect the skin surface are within a predetermined region.

10. A non-contact skin imaging device according to claim 9, wherein the collimated lights beams project points on the skin surface, and wherein the controller is arranged to judge that the skin surface is in an optimal position for capturing the photometric stereo image data if the points are spaced from each other by less than a threshold distance.

11. A non-contact skin imaging device according to claim 4, wherein the collimated light source(s) are arranged to emit a planar light beam.

12. A non-contact skin imaging device according to claim 11, wherein the collimated lights beams project lines on the skin surface, and wherein the controller is arranged to judge that the skin surface is in an optimal position for capturing the photometric stereo image data based on the position at which the lines intersect each other.

13. A non-contact skin imaging device according to claim 9, wherein the controller is arranged to determine a rate of change of the position at which each collimated light beam intersects the skin surface, whereby the controller is arranged to judge that the skin surface is in an optimal position for capturing the photometric stereo image data if the rate of change of the positions at which the collimated light beams intersect the skin surface is less than a predetermined threshold.

14. A non-contact skin imaging device according to claim 2, wherein the controller comprises a field programmable gate array in communication with the image capture device.

15. A non-contact skin imaging device according to claim 1, comprising a portable housing for supporting the photometric stereo imaging apparatus, the optical range finder and the controller.

16. A non-contact method of capturing photometric stereo image data of a skin surface, the method comprising:
determining, using an optical range finder, a position of the skin surface within a field of view of an image capture device;
judging whether or not the skin surface is in an optimal position for capturing the photometric stereo image data; and
upon judging that the skin surface is in the optimal position, automatically triggering capture of the photometric stereo image data.

17. A method according to claim 16, wherein the optical range finder comprises a plurality of collimated light sources mounted in different respective fixed positions relative to the image capture device, and wherein the method comprises:
emitting a plurality of collimated light beams through the field of view of the image capture device,
monitoring, by an image processing controller in communication with the image capture device, a position at which the collimated light beams intersect the skin surface,
wherein judging whether or not the skin surface is in an optimal position for capturing the photometric stereo image data is based on the position at which the collimated light beams intersect the skin surface.

18. A method according to claim 17, wherein judging whether or not the skin surface is in an optimal position for capturing the photometric stereo image data comprises determining whether or not the positions at which the collimated light beams intersect the skin surface are within a predetermined region.

19. A method according to claim 17, wherein the collimated lights beams project points on the skin surface, and wherein judging whether or not the skin surface is in an optimal position for capturing the photometric stereo image data comprises determining a spacing between the points.

20. A method according to claim 17, wherein the collimated lights beams project lines on the skin surface, and wherein judging whether or not the skin surface is in an optimal position for capturing the photometric stereo image data comprises determining a position at which the lines intersect each other.

21. A method according to claim 17, wherein judging whether or not the skin surface is in an optimal position for capturing the photometric stereo image data comprises determining a rate of change of the position at which each collimated light beam intersects the skin surface.
